(19) United States
(12) Patent Application Publication
(10) Pub. No.: US 2003/0113388 A1
(43) Pub. Date: Jun. 19, 2003

(54) Methods of treatment for skin disorders using turmeric extract and a hydroxy acid

(75) Inventor: Dung Phan, San Jose, CA (US)

Correspondence Address: QUINE INTELLECTUAL PROPERTY LAW GROUP, P.C., P O BOX 458, ALAMEDA, CA, 94501, US

(73) Assignee: Dung Phan

(21) Appl. No.: 10/313,912

(22) Filed: Dec. 5, 2002

Related U.S. Application Data
(60) Provisional application No. 60/341,583, filed on Dec. 13, 2001.

(51) Int. Cl. A61K 35/78

(52) U.S. Cl. 424/756

(57) ABSTRACT

This invention provides compositions and methods to treat medical disorders, such as skin conditions, using a combination of turmeric extracts, and hydroxy acids, and/or alpha-1-antitrypsin. Skin conditions such as psoriasis, severe dryness, itchiness, stretch marks, acne, and microbial infection are treated by topical application of the compositions.

### CROSS-REFERENCE TO RELATED APPLICATIONS

This application claims priority to and benefit of a prior U.S. Provisional Application No. 60/341,583, Compositions and Methods of Treatment for Skin Disorder Using Turmeric Extract by Dung Phan, filed Dec. 13, 2001. The full disclosure of these prior applications are incorporated herein by reference.

### FIELD OF THE INVENTION

The invention is in the field of methods and compositions for treating skin conditions, such as, psoriasis, severe dryness, itchiness, stretch marks, acne, and/or microbial infection by topical application of a formulation of, e.g., a turmeric extract and hydroxy acids.

### BACKGROUND OF THE INVENTION

Turmeric and turmeric extracts have long been active ingredients in compositions for topical application to treat skin diseases and conditions. Hydroxy acids are common ingredients in creams and lotions, e.g., to reduce wrinkles in skin by penetrating and/or removing the outer dry skin layers.

Curcuma longa (Fam. Zingiberaccae), or turmeric, is a spicy plant that is a common ingredient in curry powder, usually in combination with other herbs such as cayenne, garlic, cumin and onion. Turmeric has a strong yellow color that is useful in providing a pleasing color to foods such as prepared mustard. Turmeric has antimicrobial properties that can help preserve food and prevent spoilage.

Turmeric is one of the oldest herbs in Ayurveda materia medica, and has been used in Ayurveda medicine internally as a tonic, to settle an upset stomach, and as a blood purifier. Turmeric has been applied topically to wounds to stop bleeding, speed healing and reduce scaring. Ground turmeric has been used as a topical salve, particularly in Asian cultures, to prevent and treat a variety of skin diseases and conditions.

The significance of turmeric in medicine has changed in modern times with the scientific observation of turmeric's therapeutic properties. The same ground dried rhizome of Curcuma longa, which has been used for centuries as a spice, food preservative and coloring agent, has been found to be a rich source of beneficial phenolic compounds, turmerin peptide, and curcuminoids. Curcuminoids have scientifically documented anti-oxidant, anti-inflammatory, anti-bacterial, anti-fungal, antiparasitic, anti-mutagen, anti-cancer and detoxification properties. Curcuminoids are recognized for their broad biological activity and safety of use. Significant data are available on the safety, toxicity, dose range, pharmacokinetics, and other biological properties of turmeric and its components, including curcuminoids and turmerin peptide. Turmeric components have been observed to be well tolerated while providing anti-oxidant benefits, inhibit microbial growth, inhibit several enzymes, and inhibit abnormal cell growth in studies of cells, animals, and humans.

Studies have shown that turmeric components can inhibit enzymes associated with disease states. Extensive in vitro and in vivo testing has shown that turmeric inhibits chemically-induced epidermal ornithine decarboxylase activity, epidermal DNA synthesis, and the promotion of skin tumors in mice (Conney, A. H. et al., Adv. Enzyme Regul., 31:385-396, 1991; Huang, M. T. et al., Cancer Res., 48:5941-5945, 1988; Lu, Y. P. et al., Carcinogenesis, 14:293-297, 1993; Azuine, M. A., Bhide, S. V., Nutr Cancer, 17:77-83, 1992). Further studies suggest that turmeric also reduces arachidonic acid-induced rat paw and mouse skin edema and markedly inhibits epidermal lipoxygenase and cyclooxygenase activity in vitro (Rao, T. S. et al., Indian J. Med. Res., 75:574-578, 1982; Conney, A. H. et al., Adv. Enzyme Regul., 31:385-396, 1991; Huang, M. T. et al., Cancer Res., 48:5941-5945, 1988). Phosphorylation events can also be influenced by curcumin, as it has been reported that curcuminoids inhibit protein kinase C activity induced by 12-O-tetradecanoyl-phorbol-13-acetate in NIH 3T3 cells (Liu, J. Y., Lin, S. J., and Lin, J. K., Carcinogenesis, 14:857-861).

Turmeric can fight against microbial infections and parasitic infestations. In humans, ingestion of turmeric has been used to treat biliary infections where it demonstrates bacteriostatic or bacteriocidal effects against organisms involved in cholecystitis (Ramprasad, C. et al., Ind. J. Phys. and Pharm., 1:136-143, 1957; Lutumski, J. et al., Planta Med., 26:9-19, 1974). Topical application of a turmeric paste for the treatment of scabies has also shown good results (Charles, V., Charles, S. X., Trop. Geogr. Med., 44:178-181, 1992).

Their potential use of turmeric and turmeric extracts in the prevention of cancer and in the treatment of infection with human immunodeficiency virus (HIV) are the subject of intensive laboratory and clinical research. It has recently been shown that curcuminoids decreased p24 antigen production in acutely or chronically infected cells with HIV-1, a paradigm of anti-viral activity (Li, C. J. et al., Proc. Natl. Acad. Sci. USA, 90:1839-1842, 1993). The addition of turmeric to the diet has been shown to inhibit azoxymethanol-induced colonic epithelial cell proliferation and focal areas of dysplasia (Huang, M. T. et al., Cancer Letters, 64:117-121, 1992). It has also been shown to interfere with the formation of covalent carcinogen-DNA adducts (Mukudan, M. A. et al., Cardnogenesis, 14:493-496, 1993).

Fat metabolism is likewise influenced by curcumin. It can render bile non-lithogenic in mice (Hussain, M. S. et al., Indian J. Med. Rcs., 96:288-291, 1992). Curcuminoids can reduce the production of PMA-induced lipid peroxidation and 8-OH-deoxyguanosine formation in mouse fibroblast cells (Shih, C. A., and Lin, J. K., Carcinogenesis, 14:709-712, 1993). Oral administration of curcuminoids in human volunteers has been shown to significantly decrease the level of serum lipid peroxides (33%), increase HDL cholesterol (29%), and decrease total serum cholesterol (11.63%) (Soni, K, B,, Kuttanm R., Indian J. Phys. Pharmacol., 36:273-275, 1992).

Curcumin can moderate the immune system as well as smooth muscle cell proliferation. Activation responses to phytohemagglutinin and mixed lymphocyte reaction were reduced in human peripheral blood mononuclear cells in the presence of curcuminoids. Furthermore, curcuminoids inhibited the proliferation of rabbit vascular smooth muscle cells stimulated by fetal calf serum. Curcuminoids had a greater inhibitory effect on platelet derived growth factor-stimulated proliferation than on serum-stimulated proliferation (Huang, H. C. et al., Eur. J. Pharmacol., 221:381-384, 1992).

The anti-inflammatory properties of curcuminoids were shown to inhibit the 5-lipoxygenase activity in rat peritoneal neutrophils as well as the 12-lipoxygenase and the cyclooxygenase activities in human platelets (Ammon, H. P. T. et al, J. Ethopharmacol., 38:113-119, 1993). Curcuminoids had no significant effect on quercetin-induced nuclear DNA damage, lipid peroxidation and protein degradation, and thus has the unique potential of acting as both pro- and antioxidants, depending on the redox state of their biological environment (Saura, C. et al., Cancer Letters, 63:237-241, 1992). Administration of curcuminoids in mice exhibited antioxidant properties by significantly reducing the scavenging of peroxides and other activated oxygen species, (Soudamini, K. K. et al., Indian J. Phys. Pharmacol. 36:239-243, 1992).

Hydroxy acids, such as glycolic acid and lactic acid, are commonly used as ingredients in cosmetics said to reduce wrinkles, spots, and other signs of aging. The hydroxy acids are believed to provide cosmetic benefits by penetrating the dry, cornified outer layers of skin. The outer layers of skin can be defoliated in the process to reveal the softer, smoother, moister, more resilient skin beneath for a positive cosmetic benefit. Penetration with hydroxy acids can open spaces between skin cells to allow better access to lower skin layers for large molecules, hydrophobic molecules and charged molecules. Unfortunately, hydroxy acids can have undesirable side effects, including severe redness, swelling, burning, blistering, bleeding, rash, and itching and skin discoloration (Alpha Hydroxy Acids for Skin Care, U.S. food and Drug Administration, FDA Consumer, March-April, 1998).

Compositions of curcumin or turmerin with a hydroxy acid for use in treatment of scars and skin pigmentation are described in U.S. patent application Ser. No. 09/890,941 “Compositions and Methods Of Treatment For Skin Conditions Using Extracts of Turmeric” by Dung Phan, filed Aug. 6, 2001 and International Patent application PCT/US00/40641 “Compositions and Methods Of Treatment For Skin Conditions Using Extracts of Turmeric” by Dung Phan, filed Aug. 15, 2000.

Alpha-1-antitrypsin is a protein that inhibits certain proteolytic enzymes which can damage connective tissue matrices. A deficiency of alpha-1-antitrypsin has been associated with destruction of alveolar sacks by elastase enzymes in some patients with emphysema. Degradation of elastin fibers in skin may be a cause of wrinkling and drooping in aged skin. Elastase enzymes may slow the progression of would healing by damaging fibers in a newly laid connective tissue matrix. Application of alpha-1-antitrypsin in these and other instances could provide a benefit but for poor penetration through outer skin layers.

In view of the above, a need exists for methods to enhance penetration of turmeric extracts and/or alpha-1-antitrypsin. It would be desirable to have formulations of hydroxy acids that could provide the defoliant and penetratant functions without significant side effects. The present invention provides these and other features that will be apparent upon review of the following.

### SUMMARY OF THE INVENTION

The present invention provides, e.g., unique combinations of complimentary active ingredients in compositions to treat medical disorders, such as skin conditions. For example, turmeric extracts in combination with hydroxy acids can provide beneficial effects in the treatment of psoriasis, severe dryness, itchiness, stretch marks, acne, and microbial infection. In another aspect of the invention, turmeric extracts in combination with alpha-1-antitrypsin can provide beneficial effects in treatment of, e.g., psoriasis, wounds, viral infections, and wrinkles.

Methods of the invention for treating skin conditions include, e.g., application of formulations of the invention to skin having particular conditions to provide complimentary activities to effectively treat the conditions. The methods include, e.g., applying a formulation of a turmeric extract and a hydroxy acid to skin having a skin condition, such as psoriasis, severe dryness, itchiness, stretch marks, acne, or a microbial infection. An effective amount of the formulation can be applied topically to improve the skin condition. For example, a dose of the formulation can be administered to the skin one or more times per day. Doses can be administered, e.g., one, two, or more days until the desired cosmetic or therapeutic benefit is obtained. A typical dose of turmeric extracts to be administered daily to treat a typical skin condition provides, e.g., from about 0.1 ug to about 40 ug of curcuminoids or from about 0.1 ug to about 20 ug of turmerin peptide.

Turmeric extracts can be present in formulations in amounts suitable for the particular condition and method of treatment. The turmeric extracts can include, e.g., curcumin and/or turmerin. Curcumin in the formulations of the method can provide curcuminoids present in amounts ranging, e.g., from about 0.1 ug/ml to about 50 mg/ml, from about 0.1 ug/ml to about 40 ug/ml, or from about 1 ug/ml to about 20 ug/ml. Turmerin peptide in the formulations of the method can be present in amounts ranging, e.g., from about 0.1 ug/ml to about 1 mg/ml, or about 0.1 ug/ml to about 20 ug/ml.

The hydroxy acids in the formulations of the method can be, e.g., an alpha hydroxy acid, such as glycolic acid, and/or a beta-hydroxycarboxylic acid, such as salicylic acid. For example, the hydroxy acid can be glycolic acid in an amount ranging from about 0.1 weight percent to about 10 weight percent of the formulation. The hydroxy acid can be an alpha-hydroxy acid, such as, e.g., alpha-hydroxycarboxylic acids, alpha hydroxy acetic acid (h1h3glycolic acid), alpha hydroxybenzeneacetic acid (mandelic acid), alpha hydroxypropionic acid (lactic acid), alpha hydroxybutanoic acid, alpha hydroxyhexanoic acid, alpha hydroxyoctanoic acid (alpha hydroxycaprylic acid), alpha hydroxynonanoic acid, alpha hydroxydecanoic acid, alpha hydroxyundecanoic acid, alpha hydroxydodecanoic acid (alpha hydroxylauric acid), alpha hydroxytetradecanoic acid, alpha hydrocyhexadecanoic acid, alpha hydroxyoctadecanoic acid, alpha hydroxyoctaeicosanoic acid, dicarboxylic alpha hydroxy acids, dihydroxybutanedioic acid (tartaric acid), 2-hydroxybutanedioic acid (malic acid), 2-hydroxy propanedioic acid, 2-hydroxy hexanedioic acid, 2-hydroxy octanedioic acid, 2-hydroxy decanedioic acid, 2-hydroxy dodecanedioic acid, 2-hydroxy myristicdioic acid, 2-hydroxy palmiticdioic acid, Tricarboxylic alpha hydroxy acid, 2-hydroxy-1,2,3,-propanetricarboxylic acid (citric acid), and 1-hydroxy-1,2,3-propanetricarboxylic acid (isocitric acid).

The formulation of the method can include other active ingredients to provide desired properties. For example, the formulation can include vitamin B, aloe vera, or vitamin E in amounts ranging from about 0.1 weight percent to about 10 weight percent of the formulation. The formulation can include other active agents, such as, e.g., antioxidants, anticarcinogens, anti-inflammatory agents, hormones, hormone antagonists, antibiotics, and antibacterial agents to provide sysergistic or alternate effects. The formulation of the method can be provided with plants, plant parts, or plant extracts from plants such as, e.g., ginger, garlic, fenugreek, guava leaves, Aquilaria agallocha, Ficus racemosa, Saraca asoka, Trigionell foenum-graecum, Curcuma aromatica, Meriandra bengalensis, Zanthoxylum budrunga, Withania somnifera, Crocus sativus, Saussurea lappa, Eclipta alba, Bacopa monnieri, Sida retusa, Indigofera tinctoria, Cardiospermum halicacabum, and Hibiscus rosa-sinensis to contribute active and inert properties to the composition.

The formulation for topical application on skin can be blended to prepare, e.g., a cream, lotion, astringent, aerosol, salve, rub, powder, patch, and/or the like. Formulations for injection can be, e.g., sterile liquids, isotonic liquids, lyophilized cakes, spray-dried powders, and/or the like.

The formulation for use in methods to treat disorders, such as skin conditions, can include, e.g., thickeners, emulsifiers, emollients, buffers, bulking agents, penetrants, fragrances, preservatives, and/or the like, as is appreciated by those skilled in the art. For example, the formulation can include petrolatum and/or mineral oil in an amount ranging from about 1 weight percent to about 10 weight percent of the formulation. The formulation can include, e.g., dimethicone in an amount ranging from about 0.1 weight percent to about 5 weight percent of the formulation. The formulation of the invention can provide excipients, such as, e.g., salts, buffers, vegetable oils, mineral oils, benzyl alcohol, cyclodextrin, hydroxypropylcyclodextrin, polyethylene glycols, glycerol triacetate, fatty acid glycerides, gelatin, soya lecithin, carbohydrates, lactose, starch, sugars, magnesium stearate, talc, cellulose, and/or the like. The formulation can include, e.g., plant oils, such as coconut oil, sunflower oil, mustard oil, almond oil, sesame oil, safflower oil, peanut oil, and/or the like.

The present invention includes compositions for treating skin conditions. The compositions include, e.g., a turmeric extract and a hydroxy acid formulated to treat skin conditions, such as psoriasis, severe dryness, itchiness, stretch marks, acne, or microbial infection. The turmeric extract can comprise curcumin and/or turmerin. The hydroxy acid can be, e.g., glycolic acid in an amount ranging from about 0.1 weight percent to about 10 weight percent of the formulation. Other formulation ingredients can be incorporated to provide useful characteristics. For example, vitamin B, aloe vera, and/or vitamin E can be incorporated in an amount ranging from about 0.1 weight percent to about 10 weight percent of the formulation.

The present invention includes compositions containing the formulations of the invention on the skin having certain conditions. For example, the invention encompasses a composition of a turmeric extract and a hydroxy acid on human skin for treatment of skin conditions, such as psoriasis, severe dryness, itchiness, stretch marks, acne, and/or microbial infections. In this aspect of the invention, the turmeric extract can be, e.g., curcumin and/or turmerin. The hydroxy acid can be, e.g., 0.1 weight percent to about 10 weight percent glycolic acid. The composition on the skin can further provide, e.g., a vitamin B, aloe vera, or vitamin E in an amount ranging from about 0.1 weight percent to about 10 weight percent of the formulation.

### DEFINITIONS

Before describing the present invention in detail, it is to be understood that this invention is not limited to particular compositions, methods or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a surface” includes a combination of two or more surfaces; reference to “bacteria” includes mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

The term turmeric extract, as used herein, refers to a solution or suspension of molecules released from turmeric by extraction with a solvent. Turmeric extracts can include compositions such as, e.g., turmerin and/or curcumin.

The term turmerin, as used herein, refers to a turmeric extract with a hydrophilic solvent, such as aqueous solvents. Turmerin can be, e.g., a crude extract, an extract with the more hydrophobic curcuminoids substantially removed, a composition of synthetic turmerin peptide, a composition of a recombinant turmerin peptide, and/or an extract processed to provide partially or substantially purified turmerin peptide. The term turmerin peptide generally refers, herein, to the turmerin peptide constituents of a solution, suspension or dosage form, not counting, e.g., extract solvents.

The term curcumin. as used herein, refers, e.g., to a hydrophobic component of a turmeric extract. Curcumin can be, e.g., a crude extract of turmeric with a hydrophobic solvent, a crude extract of hydrophobic molecules or particles separated from an aqueous extract of turmeric, a composition of synthetic curcuminoids, or an extract processed to provide partially or substantially purified curcuminoids. The term curcuminoids generally refers, herein, to the curcuminoid constituents in a solution, suspension or dosage form, not counting, e.g., extract solvents.

The term effective amount, as used herein, refers to a quantity of formulation, active ingredient, turmeric, turmeric extract, curcumin, turmerin, curcuminoids, turmerin peptide, hydroxy acid, and/or alpha-1-antitrypsin which produces the desired effect, e.g., which treats (e.g., ameliorates or improves) the relevant disorder, such as a skin condition.

### DETAILED DESCRIPTION

The complementary (synergistic) combination of turmeric extracts and hydroxy acids provides benefits, e.g., in treating skin conditions not realized with application of the separate components. For example, the anti-oxidant, anti-inflammatory, anti-bacterial, anti-fungal, antiparasitic, anti-mutagen, and anti-cancer effects of turmeric extract formulations on skin can be significantly enhanced when the formulation includes effective amounts of hydroxy acids. Removal of cornified layers and opening of skin intercellular spaces by the hydroxy acids can, e.g., significantly improve penetration of turmeric extract components to the tissues requiring treatment. In a complimentary fashion, the anti-oxidant, anti-inflammatory, and anti-microbial effects of turmeric extracts can, e.g., reduce the irritation and inflammatory side effects of hydroxy acids, so they can provide, e.g., improved skin freshening benefits.

Alpha-1-antitrypsin (AAT) is a protein that can inhibit serine proteases, e.g., involved are involved in damage to connective tissues. However, the activity of AAT can be destroyed by certain reactive molecules, such as peroxides and radicals. The combination of turmeric extracts with alpha-1-antitrypsin (AAT) can enhance the benefits of AAT treatments by removing the reactive molecules. These benefits can be further enhanced, in topical applications, by including a hydroxy acid in the formulation to improve penetration of the turmeric/AAT combination.

Turmeric Extracts

Turmeric is a member of the family Zingiberaceae. It is generally obtained from the rhizome of the plant Curcuma loga. Turmeric can be obtained, e.g., in wholesale and retail spice markets. Curcuminoids and other molecules extractable from turmeric can also be synthesized by one of ordinary skill in the art by reference to literature on the subject. In addition, a synthetically produced curcuminoids are available, e.g., Acros Organics, Fluka and Sigma-Aldrich. Various forms of turmeric, including fresh, powdered, liquid juice, pulp, or resin, can be utilized to provide compositions such as curcumin, curcuminoids, turmerin, and turmerin peptide for use in accordance with the present invention.

Turmeric extracts can be prepared any number of ways known in the art. For example, depending on the hydrophobicity of extract solutions, soluble turmeric components can be separated into a more hydrophobic curcumin phase and a more hydrophilic turmerin phase. The curcumin phase (curcumin) can include, e.g., poorly water soluble curcuminoids, such as diferuloylmethane. The turmerin phase (turmerin) can include, e.g., turmerin peptide, a water soluble 40 amino acid anti-oxidative peptide. Commonly, turmeric extracts are, e.g., a mixture of both curcumin and turmerin.

One way to prepare extracts of turmeric is to grind the dried turmeric root, then seep the powder in hot solvents. For example, the skin of the turmeric rhizome can be removed. The turmeric can be sliced into small thin pieces. One gram of dried turmeric can be mixed with 10 mL of hot water or 20% ethanol, and the mixture can be held in the dark at room temperature for about 24 hours. The mixture can be filtered to remove depleted debris from the extract solution. The extract solution can be further clarified by centrifugation at low speed to remove particles. Aqueous phase turmeric extracts are enriched in turmerin peptide and can be, e.g., considered turmerin of this invention. Curcumin can exist, e.g., as particles and/or micelles suspended in aqueous turmeric extracts. High speed centrifugation can, e.g., separate a turmeric extract into layers of curcumin and turmerin. Under high centripetal force, a low density lipid curcumin layer can form at the top of an extract and/or a layer of higher density curcumin particles can form at the bottom of the extract. Such layers are easily harvested from the aqueous turmerin and include, e.g., curcuminoids soluble in organic solvents. Turmeric extracts of the invention can be prepared, e.g., using solvents ranging from high hydrophobicity (e.g., toluene) to low hydrophobicity (e.g., saline), or using solvents containing surfactants, to provide extracts with desired proportions of turmerin and/or curcumin components. Although the amounts of extraction products vary somewhat, guidance from analysis and from the literature support that approximately 1-5% of turmeric is curcuminoids and 0.1% is turmerin peptide. A detailed description of the biochemical features of turmerin can be found in Srinivas, L., et. al., Turmerin: A Water Soluble Antioxidant Peptide from Turmeric [Curcuma longa], Archives of Biochemistry and Biophysics, 292:617 (1992).

Turmerin, e.g., the aqueous extract of turmeric, typically includes a 5 kDa non-cyclic water soluble peptide with 3 methionine residues (turnerin peptide), chlorogenic acid, caffeoic acid, ferulic acid, and a water soluble lipid (see, e.g., Anti-oxidant activity of Curcumin and Related Compounds, Sharma Biochemical Pharmacology, Vol. 25.pp1881-1882. Pergamon Press 1976.) The turmerin peptide is, e.g., water-soluble and acts as an anti-oxidant (see, e.g., Anti-oxidant activity of Curcumin and Related Compounds, Sharma Biochemical Pharmacology, Vol. 25.pp1881-1882. Pergamon Press 1976.)

Turmerin can be formulated, e.g., with other active ingredients, excipients, bulking agents and carriers to provide benefits (see, Formulations and Indications section, below) by topical application. The concentration of turmerin peptide, as used herein, is, e.g., the amount of solids by weight in a turmeric extract aqueous solution. Turmerin is generally administered to provide a dosage of turmerin peptide ranging of from about 0.01 to 1000 ug per day, about 0.1 to 20 μg per day, or preferably about 1 to 3 μg per day.

Curcumin, e.g., the more hydrophobic extractable components of turmeric, typically includes, e.g., curcuminoids, such as curcumin I (diferuloyl methane), curcumin II (feruloyl-P-hydroxycinnamoyl methane), and curcumin III (bis-(P-hydroxycinnamoyl) methane). These curcumin components can dissolve, e.g., into organic solvents. More commonly, in the present invention, curcumin exists, e.g., as a colloid suspension of micelles, or as hydrophobic particles, within an aqueous extract, such as turmerin. Optionally, curcumin of the invention can exist, e.g., as a waxy paste of hydrophobic particles when separated from turmeric extracts by centrifugation, filtration and/or hydrophobic adsorption.

The turmeric extract curcumin provides scavenging and inhibitory effects that are complimentary to activities of other formulation ingredients, as is discussed below. Curcuminoids are able to scavenge superoxide radicals as discussed in Free Radical Scavenging Activity of Curcumoids, by Sreejayan, N. and Rao, M. N., Arzneimittelforschung 46(2): 169-71, 1996. Curcuminoids are scavengers of nitric oxide, as discussed in Nitric Oxide Scavenging by Curcumoids, by Sreejayan, Rao, M. N., J Pharm Pharmacol, 49(1): 105-7, 1997. Curcuminoids are also potent inhibitors of arachidonic acid induced inflammation, as is discussed in Turmerin: a Water Soluble Antioxidant Peptide from Turmeric, by. Srinivas, L., Shalini, V. K. and Shylaya, M., Arch Biochem Biophys 292(2): 617-23, 1992. Curcuminoids are considered to be antioxidants and anti-inflammation agents, (see, Concise Encyclopedia of Chemistry, Walter de Gruyter Berlon-New York 1994-Page 1161-second edition.

Curcumin can be formulated, e.g., with other active ingredients, excipients, bulking agents and carriers to provide benefits (see, Formulations and Indications section, below) by topical application. The concentration of curcuminoids, as used herein, is, e.g., the amount of curcuminoids and other lipid-like turmeric extract components by weight in a composition or formulation. Curcuminoids are generally administered in a range of from about 0.1 ug to about 10 mg or 50 mg per day, about 0.1 ug to 40 ug per day, or about 2 ug to 6 ug per day. In one treatment regime, about 2 ug to 3 ug of curcuminoids is administered per day until the condition is ameliorated.

Hydroxy Acids

Hydroxy acids, in the compositions and methods of the invention, compliment the activities of other formulation ingredients, e.g., by enhancing penetration to the treated tissues. In addition, in many embodiments of the invention, hydroxy acids provide, e.g., cosmetic benefits to the skin.

Alpha- and beta-hydroxy acids ranging, e.g., from C₂ to C₃₀ are suitable for the present invention. The preferred beta-hydroxycarboxylic acids are primarily exemplified by salicylic acid and C₁ to C₃₀ ester and salt derivatives. Examples of suitable alpha-hydroxycarboxylic acids include but are not limited to: alpha hydroxy acetic acid (h1h3glycolic acid); alpha hydroxybenzeneacetic acid (mandelic acid); alpha hydroxypropionic acid (lactic acid); alpha hydroxybutanoic acid; alpha hydroxyhexanoic acid; alpha hydroxyoctanoic acid (alpha hydroxycaprylic acid); alpha hydroxynonanoic acid; alpha hydroxydecanoic acid; alpha hydroxyundecanoic acid; alpha hydroxydodecanoic acid (alpha hydroxylauric acid); alpha hydroxytetradecanoic acid; alpha hydrocyhexadecanoic acid; alpha hydroxyoctadecanoic acid; alpha hydroxyoctaeicosanoic acid; dicarboxylic alpha hydroxy acids; dihydroxybutanedioic acid (tartaric acid); 2-hydroxybutanedioic acid (malic acid); 2-hydroxy propanedioic acid; 2-hydroxy hexanedioic acid; 2-hydroxy octanedioic acid; 2-hydroxy decanedioic acid; 2-hydroxy dodecanedioic acid; 2-hydroxy myristicdioic acid; 2-hydroxy palmiticdioic acid; tricarboxylic alpha hydroxy acid; 2-hydroxy-1,2,3,-propanetricarboxylic acid (citric acid); 1-hydroxy-1,2,3-propanetricarboxylic acid (isocitric acid), and mixtures thereof.

C₁ to C₃₀ esters and salts of alpha- and beta-hydroxycarboxylic acids (e.g. potassium, sodium, ammonium, triethanolammonium salts) are also meant to be included within the term “alpha- and beta-hydroxycarboxylic acid”. Depending on the pH of the composition, a mixture of the salt and the acid may be present, as is appreciated by those skilled in the art.

Preferred alpha hydroxycarboxylic acids include monocarboxylic acids, in order to improve skin penetration and efficacy. In one class of embodiments, the hydroxy acid is chosen from lactic acid, H₂H₄glycolic acid, mandelic acid, and mixtures thereof to optimize the efficacy of compositions by increasing percutaneous absorption. Most preferred is the L-form of an alpha hydroxycarboxylic acid.

Alpha-1-Antitrypsin

Alpha-1-antitrypsin (AAT) is a 45 kDa protein that can inhibit the activity of certain serine protease enzymes. AAT can be secreted into the bloodstream by the liver to protect tissues from degradation, for example, by neutrophil cell elastase during inflammatory responses. Inhibition of serine protease activity by AAT can provide, e.g., cosmetic and/or therapeutic benefits when administered in the compositions and methods of the invention, as described in the Formulations and Indications section, below.

AAT can be obtained, e.g., by purification from natural sources or from recombinant sources. For example, ATT can be purified from pooled human plasma by a suitable combination of purification methods known in the art, such as precipitation, centrifugation, immunoaffinity capture, ion exchange chromatography, size exclusion chromatography, hydrophobic interaction chromatography, ultrafiltration, diafiltration, and/or the like. Alternately, AAT can be obtained, e.g., by incorporation of an AAT DNA sequence into an expression vector to provide a recombinant AAT protein.

Turmeric Extract Formulations and Indications for Use

Turmeric extracts are known to provide a variety of useful properties, such as, e.g., anti-oxidation, anti-inflammation, anti-microbial, antiparasitic, anti-mutagen, anti-cancer, and detoxification effects. Complimentary cosmetic and/or therapeutic effects can be realized on administration of turmeric extracts in combination with other active agents, such as, e.g., hydroxy acids and/or AAT.

Turmeric extracts in combination with hydroxy acids, for example, can provide benefits on administration for indications such as psoriasis, severe skin dryness, itchiness, stretch marks, acne, microbial infections, and/or the like. Turmeric extracts in combination with AAT can help treat, e.g., wounds, skin wrinkles, viral infections, psoriasis, and the like. The formulations described herein can be utilized, e.g., in both human and veterinary medicine.

Compositions of the invention can be administered through a variety of routes known in the art, such as oral, injection, topical application, inhalation, and/or the like. In many cases, topical application provides clear benefits such as ease of delivery, lower purification requirements for active ingredients, less stringent regulatory requirements, and the ability to target treatment directly to specific affected sites.

The following provides descriptions of indications for use of methods and compositions of the invention. Without being bound to any particular theory, e.g., mechanisms of pathology and treatment are presented based on references cited herein, the inventor's research, and the inventor's belief.

The formulations of the invention can include, e.g., bulking agents, thickeners, emollients, carriers, penetrants, stabilizers, and/or the like. Although a particular active or inert ingredient of a formulation is described as providing a particular characteristic, the ingredient can concomitantly, e.g., provide other unstated effects and/or characteristics to the formulation. For example, an emollient can also be a thickener, a hydroxy acid can act as a penetrant and a defoliant, and turmeric extracts can act as enzyme inhibitors, antioxidants, and more.

Formulations in General

Formulations of the invention generally include, e.g., active and inert ingredients mixed into a neat or diluted turmeric extract. Some ingredients can require special handling, such as pH adjustment, heating, dissolution in special solvents, and the like, as is known in the art, before addition to the formulation. The formulations of the present invention can be administered alone, or they can be mixed with a pharmaceutically or cosmetically acceptable carrier or diluent.

Formulations for topical application to skin can generally be formulated by, e.g., adding from about 10 mg to about 1 g of a hydroxy acid, and/or from about 100 mg to about 500 mg of AAT to turmeric extract in a 10 ml volume. The 10 ml formulation can also, incorporate, e.g., from about 10 mg to about 1 g of vitamin B, aloe vera and/or vitamin E. The 10 ml formulation can incorporate, e.g., from about 100 mg to about 1 g of petrolatum and/or mineral oil. The formulation can include, e.g., from about 10 mg to about 500 mg of dimethicone per 10 ml final volume.

Alpha and beta hydroxy acids, including, but not limited to, glycolic acid and salicylic acid, can be used in relatively high concentrations for compositions and methods of the present invention, due to the protective aspects of turmeric extracts. However, care should be taken to when high concentrations are to be applied to a patient, particularly in areas with sensitive skin. An approach of gradual concentration escalation can be used to avoid adverse reactions in some patients. In compositions of the present invention, an alpha hydroxy acid concentration of between approximately 0.01% and 20% is contemplated, preferably between approximately 0.1% and 10%, more preferably between approximately 1% and 5%.

Turmeric extract formulations can be prepared with excipients adapted to provide, e.g., appropriate texture, stability, pH, rheological properties, and the like, depending on the particular application. Excipients for the formulation can include, e.g., organic and inorganic substances which are appropriate for enteral, parenteral, or oral administration, e.g., water, saline, buffers, vegetable oils, mineral oils, benzyl alcohol, cyclodextrin, hydroxypropylcyclodextrin (especially beta-type), polyethylene glycols, glycerol triacetate and other fatty acid glycerides, gelatin, soya lecithin, carbohydrates such as lactose or starch or other sugars, magnesium stearate, talc, and/or cellulose. The preparations can be sterilized and/or contain additives, such as preservatives or stabilizers. Turmeric can be formulated with various oils, including coconut, sunflower, mustard, almond, sesame, safflower, peanut and/or the like.

Turmeric extracts and hydroxy acids can be formulated with various active agents, such as, e.g., vitamins, nutrients, herbs, plant extracts, and/or the like. The formulation can include B vitamins, vitamin E, tocopheryl acetate and/or calcium, for example. Turmeric and/or turmeric extracts can also be formulated with other herbs, plants, and extracts, depending on the desired purpose and effect. These herbs and plants can include, e.g., ginger, garlic, fenugreek, guava leaves, Aquilaria agallocha, Ficus racemosa, Saraca asoka, Trigionell foenum-graecum, Curcuma aromatica, Meriandra bengalensis, Zanthoxylum budrunga, Withania somnifera, Crocus sativus, Saussurea lappa, Eclipta alba, Bacopa monnieri, Sida retusa, Indigofera tinctoria, Cardiospermum halicacabum, Hibiscus rosa-sinensis, and/or the like. Other plants and herbs that can be beneficially combined in the formulations of the invention include those mentioned in various text and publications, e.g., E. S. Ayensu, Medicinal Plants of West Africa, Reference Publications, Algonac, Mich. (1978); P. Back, The Illustrated Herbal 1987, Hamlyn Publishers, distributed by Octopus Books, Printed in Hong Kong by Mandarin, ISBN 0-600 553 361; F. Bianchini and F. Corbetta, The Fruits of the Earth, translated from Italian by A. Mancinelli, Bloomsbury Books, London, ISBN 1-870630-10-6; H. M. Burkill, The Useful plants of West Tropical Africa, Ed. 2, V. I, Royal Botanic Gardens Kew, ISBN 0-947643-01-X (1985); L. Boulos, Medicinal Plants of North Africa, Reference Publications Inc., Algonac, Mich. (1983); and N. C. Shah, Herbal Folk Medicines in Northern India, J. Ethnopharm, 6:294-295 (1982).

Turmeric extracts and hydroxy acids can also be formulated into topical preparations with commonly used pharmaceutically acceptable non-toxic carriers. A formulation effective in treating many skin diseases or conditions comprises curcumin, glycolic acid, a cream base, vitamins C and E, dimethicone, vitamin B5, and salicylic acid. Other ingredients that can be combined in useful topical formulations include, e.g., sodium lauryl sulfate, oleanic acid, linoleamide DEA, glycol stearate, stearic acid, sodium hydroide, trisodium EDTA, tetrasodium EDTA, alcohols, polyethylene glycols, fragrances, preservatives, deionized water, glycerin, antibacterials, and other ingredients that are appreciated by one of skill.

The turmeric extracts and hydroxy acids can also be co-administered with other active agents to achieve synergistic effects. For example, the turmeric can be co-administered with an antibiotic for the treatment of any of the above-mentioned disorders to prevent or treat bacterial infections. Other useful active agents include, e.g., antioxidants, vitamins A and E, anticarcinogens, anti-inflammatory agents, hormones and hormone antagonists, antiseptics, and other medically useful ingredients, such as those identified in, e.g., Remington's Pharmaceutical Sciences, Eighteenth Edition, Mack Publishing Company, 1990.

Psoriasis

Psoriasis is, e.g., a persistent skin disease presenting inflammation with redness and thickened areas often on the scalp, elbows, knees, and/or lower back. The cause of psoriasis is still unclear, but at a molecular level phosphorylase kinase II and elastase appear to play roles. Turmeric is known to selectively inhibit the activity of phosphorylase kinase and can thus, help alleviate psoriasis. Formulations of the invention for treatment of psoriasis can include, e.g., AAT to inhibit elastase activity. In such formulations, turmeric extracts can play multiple roles including prevention of AAT inactivation by free radicals and other factors. Hydroxy acids can be included in the formulations, e.g., to help other active ingredients penetrate the skin surface to reach the inflamed tissues below.

Turmeric extract can be formulated into a composition for topical application to fight inflammation and to promote healing in psoriasis by, e.g., inhibition of phosphorylase kinase II activity, anti-inflammatory activity, AAT protectant activity, and general tissue-repairing/regeneration activities. Useful formulations for treatment of psoriasis include turmerin and/or curcumin in combination with, e.g., 1 to 10 weight percent hydroxy acid, and/or 1 to 5 weight percent AAT. A preferred hydroxy acid is glycolic acid. The formulation for treatment of psoriasis can also include 1 to 10 weight percent pure petrolatum and/or mineral oil, 0.5 to 5 weight percent dimethicone, 0.1 to 10 weight percent panthenol (vitamin B), and/or 0.1 to 10 weight percent aloe vera.

The formulation can be applied, e.g., topically to the areas of skin affected by psoriasis. For example, the formulation can be a salve that includes excipients, such as oils or dimethicone, that, e.g., reduce friction for rubbing the formulation into the skin, thicken the formulation so it does not run off the skin surface, and retain the formulation in a semi liquid state so diffusion of ingredients can continue over a long period of time.

The formulation for treatment of psoriasis can be applied in an effective amount to alleviate the symptoms. For example, the formulation can be applied once or twice daily over affected areas. The dose of formulated curcuminoids applied to each square inch of affected skin per day can range, e.g., from about 1 ug to about 50 mg, or 10 mg. The dose of formulated hydroxy acid applied to each square inch of affected skin per day can range, e.g., from about 1 mg to about 100 mg. The dose of formulated AAT applied to each square inch of affected skin per day can range, e.g., from about 1 mg to about 50 mg.

Severe Skin Dryness and Itching

Oxidants, loss of lipids and inflammation can be causes of dry and itchy skin. Turmeric extracts have anti-oxidant and anti-inflammatory aspects that can neutralize the source of the problem to sooth many dry and itchy skin conditions. Furthermore, lipid ingredients of the formulation, including curcuminoids, can soften the skin, form a protective layer and help seal in moisture. Hydroxy acids can contribute to the treatment by aiding penetration of the turmeric extracts and by exfoliating dry, scaly skin.

Turmeric extracts can be formulated into a topical cream or lotion to with emollients, anti-oxidants and protectants to treat dry and itchy skin disorders. Useful compositions include, e.g., turmeric extracts formulated with from about 1 to 10 weight percent hydroxy acid, from about 1 to 10 weight percent petrolatum and/or mineral oil, from about 0.1 to 5 weight percent dimethicone, from about 0.1 to 10 weight percent panthenol (Vit B 5), and/or from about 0.1 to 10 weight percent aloe vera.

The formulation for treatment of dry/itchy skin can be applied in an effective amount to alleviate the symptoms. For example, the formulation can be applied once or twice daily over affected areas. The dose of formulated curcuminoids applied to each square inch of affected skin per day can range, e.g., from about 1 ug to about 10 mg. The dose of formulated hydroxy acid applied to each square inch of affected skin per day can range, e.g., from about 1 mg to about 100 mg.

Stretch Marks

Stretch marks are scar-like manifestations visible on skin that has changed surface area due to, e.g., pregnancy, weight loss, or muscle building. The mechanism of stretch mark formation is not well understood but may involve phenomena surrounding the reconfiguration of connective tissue matrix components, such as elastin or collagen fibers, during changes in skin surface area. An accumulation of phosphorylase kinase activity in the affected area has been correlated with formation of stretch marks. Blocking activity of the kinase can help restore affected skin to a normal appearance.

Turmeric is known to be a selective inhibitor of phosphorylase kinase. Turmeric extracts, curcumin in particular are known to promote healing by speeding tissue regeneration processes. These properties of turmeric extracts, in combination with penetration enhancement by hydroxy acids, can provide formulations for reducing the appearance of stretch marks on skin.

Turmeric extracts can be formulated with hydroxy acids and other ingredients to treat and prevent the appearance of stretch marks. For example, neat or diluted turmeric extracts can be formulated by addition of about 0.1 to 10 weight percent hydroxy acid, such as glycolic acid, about 1 to 5 weight percent vitamin E, about 1 to 10 weight percent vitamin B, about 1 to 5 weight percent dimethicone, and/or about 0.1 to 10 weight percent aloe vera. These active ingredients can be blended into a conventional cream/lotion as a carrier for topical administration.

The formulation for treatment of stretch marks can be applied in an effective amount to reduce the appearance of stretch marks in affected areas. For example, the formulation can be applied once or twice daily over affected areas. The dose of formulated curcuminoids applied to each square inch of affected skin per day can range, e.g., from about 1 ug to about 10 mg. The dose of turmerin peptide applied to each square inch of affected skin per day can range, e.g., from about 0.1 ug to about 10 mg, or about 1 ug to about 1 mg. The dose of formulated hydroxy acid applied to each square inch of affected skin per day can range, e.g., from about 1 mg to about 100 mg.

Acne Treatment

There are numerous factors involved in Acne formation. For example, bacteria can get inside the pores on the surface of skin, causing infections in the pores. Oils and fats in the sebum can oxidize and thicken to inhibit the normal flow of sebaceous gland secretions. Inflammation from gland clearing processes or bacterial infections can cause swelling and reddening. These and other factors can lead to a break out of acne on the surface of skin.

The formulations of the invention can help prevent and treat acne on several fronts. The compositions of the invention can inhibit microbial infections, prevent oxidation of sebaceous secretions, defoliate skin to unblock pores, reduce inflammation, and promote healing. To prevent and treat acne, the skin can be cleaned of dirt and excess oils before application of formulations of the invention containing turmeric extracts and hydroxy acids. Previously broken skin surfaces can heal rapidly with microbial invaders inhibited or killed. Pore blockages can be removed and inflammation reduced to prevent recurrence of the acne.

Turmeric extract can be formulated, e.g., into a conventional cream, lotion, astringent, or gel carrier to treat and prevent acne and white/black head formation on the skin. Exemplary formulations for prevention or treatment of acne include, e.g. neat or diluted turmeric extract combined with about 0.1 to 10 weight percent of a hydroxy acid, such as glycolic acid, about 0.1 to 10 weight percent vitamin E, about 0.1 to 10 weight percent vitamin B, and/or 0.1 to 5 weight percent dimethicone.

The formulation for treatment of acne can be applied in an effective amount to reduce treat and/or prevent acne on the skin. For example, the formulation can be applied once or twice daily over affected areas. The dose of curcuminoids applied in the formulation to each square inch of affected skin per day can range, e.g., from about 1 ug to about 10 mg. The dose of turmerin peptide applied to each square inch of affected skin per day can range, e.g., from about 1 ug to about 10 mg, or about 1 mg. The dose of formulated hydroxy acid applied to each square inch of affected skin per day can range, e.g., from about 1 mg to about 100 mg.

Microbial Infections

Turmeric extract can be formulated with hydroxy acids to treat, e.g., bacterial and fungal infections of the skin. Such formulations can, e.g., battle the infection on many fronts. Turmeric extracts are known to inhibit and/or kill various pathogenic bacteria and fungi. Hydroxy acids can provide an environment, e.g., of low pH and high ionic strength not conducive to growth of microbes. The anti-inflammation, wound healing, penetrating, and skin cleansing properties of many formulations of the invention can also help treat microbial infections of the skin.

Turmeric extract can be formulated, e.g., into a conventional cream, lotion or gel carrier to treat microbial infections on the skin. Formulations for treating skin infections can be, e.g., a dry powder or salve to help reduce the availability of moisture to the microbes. Exemplary formulations for treatment of skin infections include, e.g. neat, dried, or diluted turmeric extract combined with about 0.1 to 10 weight percent of a hydroxy acid, such as glycolic acid, about 0.1 to 10 weight percent vitamin E, about 0.1 to 10 weight percent vitamin B, such as vitamin B5, and/or 0.1 to 5 weight percent dimethicone.

The formulation for treatment of skin infections can be applied in an effective amount to treat and/or prevent infections on the skin. For example, the formulation can be applied once or twice daily over affected areas. The dose of formulated curcuminoids applied in the formulation to each square inch of affected skin per day can range, e.g., from about 1 ug to about 10 mg. The dose of turmerin peptide applied to each square inch of affected skin per day can range, e.g., from about 1 ug to about 1 mg. The dose of formulated hydroxy acid applied to each square inch of affected skin per day can range, e.g., from about 1 mg to about 100 mg.

Wound Healing

The process of wound healing after, e.g., a traumatic injury or surgery, is a complex process progressing from blood clotting, inflammation and intrusion of immune system white cells, migration of immature connective tissue cells into the region, laying down of a connective tissue matrix, closure of the wound, and scarification or regeneration of a normal tissue structure. Wound healing progress can be impeded by, e.g., infection, excessive proteases, cell growth inhibition, toxin build up, and excessive inflammation. Formulations of the invention comprising turmeric extract, and alpha-1-antitrypsin, and/or hydroxy acids, can work against factors that slow healing.

The formulation components of the invention work as a complimentary wound healing system. For example, while the serine protease inhibitor AAT prevents destruction of newly established connective tissue fibers, turmeric extracts protect the AAT from damage by free radicals, and hydroxy acids help other active ingredients penetrate to tissues to be treated. In addition to protecting AAT, the turmeric extract component can, e.g., reduce oxidation of new tissues and inhibit growth of microbes. Application of turmeric/AAT formulations can accelerate wound healing and minimize the formation of scar tissue.

Turmeric extract can be formulated, e.g., into a conventional solution, cream, lotion or gel carrier to treat wounds. Formulations for treating wounds can be, e.g., a dry powder or salve to help reduce the availability of moisture to the microbes. Exemplary formulations for treatment of wounds include, e.g. neat, dried, or diluted turmeric extract combined with about 0.1 to 5 weight percent AAT, 0.1 to 10 weight percent of a hydroxy acid, such as glycolic acid, about 0.1 to 10 weight percent vitamin E, about 0.1 to 10 weight percent vitamin B, and/or 0.1 to 5 weight percent dimethicone.

The formulation for treatment of wounds can be applied in an effective amount to treat wounds on or beneath the skin. For example, the formulation can be applied once or twice daily over affected areas. The dose of formulated curcuminoids applied in the formulation to each square inch of affected skin per day can range, e.g., from about 1 ug to about 10 mg. The dose of turmerin peptide applied to each square inch of affected skin per day can range, e.g., from about 1 ug to about 10 mg, or about 1 mg. The dose of formulated AAT applied to each square inch of affected skin per day can range, e.g., from about 1 mg to about 50 mg. The dose of formulated hydroxy acid applied to each square inch of affected skin per day can range, e.g., from about 1 mg to about 100 mg.

Treatment of Viral Infections

Recent discoveries indicate that Alpha One Anti-Trypsin (AAT) can effectively inhibit HIV infection of cells in vitro by binding to the virus surface receptor and by inhibiting nuclear transcription factor NF kappa B production necessary for viral replication. NF kappa B activity can also be inhibited by antioxidants such as curcuminoids. AAT can also prevent, e.g., viral replication, assembly, dispersion, and attachment by inhibiting serine proteases involved in those processes. However, as described above, AAT has reduced effectiveness in the presence of free radicals. Again, the combination of formulation ingredients of this invention compliment each other to provide effective compositions for treatments. For example, while AAT inhibits viral replication and associated cell division and inflammation, turmeric extracts can provide antioxidant effects that protect AAT while inhibiting NF kappa B activity.

For administration in treatment of viral diseases of the skin, AAT and turmeric extracts can be formulated, e.g., as a solution, cream, lotion, or salve for topical application with or without hydroxy acids. Effective concentrations of some active ingredients can be obtained, e.g., by diffusion through the skin to body fluids from topically applied formulations, especially when using hydroxy acids to aid in penetration. However, AAT and turmeric extract can be more readily delivered by injection or infusion of sterile solutions for treatment of HIV and other viral infections. Formulations for injection can be prepared, e.g., by lyophilization of purified turmeric extracts and AAT along with bulking agents and protectants, such as buffers and mannitol, as is known in the art. Lyophilized formulations can, e.g., store well and be reconstituted with sterile water or saline before use.

The formulation for treatment of viral infections can be applied in an effective amount to inhibit the virus or mitigate symptoms of the infection. For topical administration, for example, the formulation can be applied once or twice daily over large skin surfaces and/or over affected areas. The dose of formulated curcuminoids applied in the formulation to each square inch of affected skin per day can range, e.g., from about 1 ug to about 100 mg. The dose of turmerin peptide applied to each square inch of affected skin per day can range, e.g., from about 1 ug to about 10 mg. The dose of formulated AAT applied to each square inch of affected skin per day can range, e.g., from about 1 mg to about 50 mg. The dose of formulated hydroxy acid applied to each square inch of affected skin per day can range, e.g., from about 1 mg to about 200 mg. For administration by infusion or injection, an effective dose of turmeric extract and AAT can be provided for treatment of the patient. For example, from about 0.1 mg to about 10 mg of curcuminoids, and about 0.1 mg to about 10 mg of AAT can be infused into a patient per kilogram per day.

Wrinkles on the Skin

Below the skin surface, there is a region called the dermal-epidermal Junction (DEJ), which is about 100 nm thick. The DEJ is the interface between the dermis and epidermis both physically and functionally. For example, the DEJ is involved in a number of biological processes, such as tissue repair, tissue attachment, migration, proliferation, and differentiation of epidermis cells. Thus, the DEJ is a physical and chemical barrier responsible for cohesion between dermis and epidermis and resistance to external traction forces on the skin.

This cohesion results from the presence of anchoring molecules in the DEJ, e.g., laminins, integrins, collagen type IV and VII, and other proteins specific to the DEJ. Since the DEJ governs the surface state of the skin, if it is folded, the surface of the DEJ increases, favoring exchanges between the dermis and epidermis, reinforcing the cohesion between the two zones. If the DEJ flattens with age or is damaged, exchanges are attenuated and the two layers are less cohesive. This results in the skin being less firm and tight leading to sagging skin and wrinkles.

A major destructive force on the DEJ is elastase, which attacks collagen and elastin in the DEJ, particularly when the ratio between elastase and its prime biological inhibitor, alpha-1-antitrypsin (AAT), is unfavorably high. Elastase can play a productive role in the skin by disassembling oxidized connective tissue fibers so new ones can be laid down. However, excessive proteolysis by elastin can damage the cohesion and anchoring molecules in the DEF resulting in wrinkles. AAT is normally present in the dermis and epidermis to moderate the activity of a serine proteases, such as elastin. An AAT deficiency can be caused, e.g., by genetic disorders and/or free radical formation that inactivates AAT. Without inhibition from AAT, elastase actively can destroy collagen and elastin to damage the DEJ (as well as connective tissue structures in other organs, such as the lungs).

To combat skin aging and premature appearance of wrinkles, AAT and turmeric extract can be formulated together into a synergistic formulation. Turmeric extracts have been known as antioxidant agents that can reduce the oxidation, and loss of flexibility, of connective tissue fibers to prevent premature skin wrinkling. The present invention provides a complimentary combination of turmeric extracts with AAT to not only prevent oxidation but to inhibit enzymatic degradation of connective tissue. Turmeric extracts can provide their antioxidant benefits, while, e.g., they protect AAT inactivation by free radicals. Thus the DEJ proteins are protected from both oxidation and enzymatic degradation. Effectiveness of the formulation can be further enhanced, e.g., by addition of hydroxy acids to the formulation to increase penetration of other active ingredients to the DEJ (while also providing a refreshed skin surface by defoliation). The unique formulations of the invention can, e.g., provide younger looking skin than for formulations without the combination.

Turmeric extract can be formulated, e.g., into a conventional solution, cream, lotion, or gel carrier to treat wrinkles. Exemplary formulations for prevention and/or treatment of wrinkles on the skin include, e.g. neat, dried, or diluted turmeric extract combined with about 0.1 to 5 weight percent AAT, 0.1 to 10 weight percent of a hydroxy acid, such as glycolic acid, about 1 to 5 weight percent vitamin E, about 1 to 10 weight percent vitamin B, about 0.1 to 10 weight percent aloe vera, and/or about 1 to 5 weight percent dimethicone.

The formulation for treatment of wrinkles can be applied in an effective amount to treat and/or prevent wrinkles on the skin. For example, the formulation can be applied once or twice daily over affected areas. The dose of curcuminoids applied in the formulation to each square inch of affected skin per day can range, e.g., from about 1 ug to about 10 mg. The dose of turmerin peptide applied to each square inch of affected skin per day can range, e.g., from about 1 ug to about 10 mg, or about 1 mg. The dose of formulated AAT applied to each square inch of affected skin per day can range, e.g., from about 1 mg to about 50 mg. The dose of formulated hydroxy acid applied to each square inch of affected skin per day can range, e.g., from about 1 mg to about 100 mg.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

While the foregoing invention has been described in some detail for purposes of clarity and understanding, it will be clear to one skilled in the art from a reading of this disclosure that various changes in form and detail can be made without departing from the true scope of the invention. For example, all the compositions, techniques and methods described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of treating skin conditions, wherein the method comprises:
applying a formulation to skin;
wherein the formulation comprises a turmeric extract and a hydroxy acid; and,
wherein the skin comprises a skin condition selected from the group consisting of psoriasis, severe dryness, itchiness, stretch marks, acne, and microbial infection.

2. The method of claim 1, wherein applying comprises topically administering an effective amount of the formulation to improve the skin condition.

3. The method of claim 1, wherein applying comprises topically administering a dose of the formulation to the skin one or more times per day.

4. The method of claim 3, wherein applying further comprises administering the formulation to the skin for two or more days.

5. The method of claim 3, wherein a total dose administered each day comprises from about 0.1 ug to about 50 mg of curcuminoids or from about 0.1 ug to about 1 mg of turmerin peptide.

6. The method of claim 5, wherein a total dose administered each day comprises from about 0.1 ug to about 40 ug of curcuminoids or from about 0.1 ug to about 20 ug of turmerin peptide.

7. The method of claim 1, wherein the turmeric extract comprises curcumin or turmerin.

8. The method of claim 7, wherein the curcumin is present in the formulation to provide curcuminoids in an amount ranging from about 0.1 ug/ml to about 50 mg/ml.

9. The method of claim 8, wherein the curcumin is present in the formulation to provide curcuminoids in an amount ranging from about 0.1 ug/ml to about 40 ug/ml.

10. The method of claim 8, wherein the curcumin is present in the formulation to provide curcuminoids in an amount ranging from about 1 ug/ml to about 20 ug/ml.

11. The method of claim 7, wherein the turmerin is present in the formulation in an amount providing turmerin peptide in an amount ranging from about 0.1 ug/ml to about 1 mg/ml.

12. The method of claim 11, wherein the turmerin is present in the formulation in an amount providing turmerin peptide in an amount ranging from about 0.1 ug/ml to about 20 ug/ml.

13. The method of claim 1, wherein the hydroxy acid is an alpha-hydroxy acid selected from the group consisting of alpha-hydroxycarboxylic acids, alpha hydroxy acetic acid (h1h3glycolic acid), alpha hydroxybenzeneacetic acid (mandelic acid), alpha hydroxypropionic acid (lactic acid), alpha hydroxybutanoic acid, alpha hydroxyhexanoic acid, alpha hydroxyoctanoic acid (alpha hydroxycaprylic acid), alpha hydroxynonanoic acid, alpha hydroxydecanoic acid, alpha hydroxyundecanoic acid, alpha hydroxydodecanoic acid (alpha hydroxylauric acid), alpha hydroxytetradecanoic acid, alpha hydrocyhexadecanoic acid, alpha hydroxyoctadecanoic acid, alpha hydroxyoctaeicosanoic acid, dicarboxylic alpha hydroxy acids, dihydroxybutanedioic acid (tartaric acid), 2-hydroxybutanedioic acid (malic acid), 2-hydroxy propanedioic acid, 2-hydroxy hexanedioic acid, 2-hydroxy octanedioic acid, 2-hydroxy decanedioic acid, 2-hydroxy dodecanedioic acid, 2-hydroxy myristicdioic acid, 2-hydroxy palmiticdioic acid, Tricarboxylic alpha hydroxy acid, 2-hydroxy-1,2,3,-propanetricarboxylic acid (citric acid), and 1-hydroxy-1,2,3-propanetricarboxylic acid (isocitric acid).

14. The method of claim 1, wherein the hydroxy acid is a beta-hydroxycarboxylic acid or salicylic acid.

15. The method of claim 1, wherein the hydroxy acid comprises glycolic acid in an amount ranging from about 0.1 weight percent to about 10 weight percent of the formulation.

16. The method of claim 1, wherein the formulation further comprises a vitamin B, aloe vera, or vitamin E in an amount ranging from about 0.1 weight percent to about 10 weight percent of the formulation.

17. The method of claim 1, wherein the formulation further comprises petrolatum or mineral oil in an amount ranging from about 1 weight percent to about 10 weight percent of the formulation.

18. The method of claim 1, wherein the formulation further comprises dimethicone in an amount ranging from about 0.1 weight percent to about 5 weight percent of the formulation.

19. The method of claim 1, wherein the formulation further comprises excipients selected from the group consisting of water, saline, buffers, vegetable oils, mineral oils, benzyl alcohol, cyclodextrin, hydroxypropylcyclodextrin, polyethylene glycols, glycerol triacetate, fatty acid glycerides, gelatin, soya lecithin, carbohydrates, lactose, starch, sugars, magnesium stearate, talc, and cellulose.

20. The method of claim 1, wherein the formulation further comprises plant oils selected from the group consisting of coconut oil, sunflower oil, mustard oil, almond oil, sesame oil, safflower oil, and peanut oil.

21. The method of claim 1, wherein the formulation further comprises plants selected from the group consisting of ginger, garlic, fenugreek, guava leaves, Aquilaria agallocha, Ficus racemosa, Saraca asoka, Trigionell foenum-graecum, Curcuma aromatica, Meriandra bengalensis, Zanthoxylum budrunga, Withania somnifera, Crocus sativus, Saussurea lappa, Eclipta alba, Bacopa monnieri, Sida retusa, Indigofera tinctoria, Cardiospermum halicacabum, and Hibiscus rosa-sinensis.

22. The method of claim 1, wherein the formulation further comprises active agents selected from the group consisting of antioxidants, anticarcinogens, anti-inflammatory agents, hormones, hormone antagonists, antibiotics, and antibacterial agents.

23. A composition for treating skin conditions, the composition comprising:
a turmeric extract; and,
a hydroxy acid;
wherein the composition is formulated to treat a skin condition comprising psoriasis, severe dryness, itchiness, stretch marks, acne, or microbial infection.

24. The method of claim 23, wherein the turmeric extract comprises curcumin or turmerin.

25. The method of claim 23, wherein the hydroxy acid comprises glycolic acid in an amount ranging from about 0.1 weight percent to about 10 weight percent of the formulation.

26. The method of claim 23, wherein the formulation further comprises a vitamin B, aloe vera, or vitamin E in an amount ranging from about 0.1 weight percent to about 10 weight percent of the formulation.

27. A composition on the skin of a human for treatment of a skin condition, the composition comprising:
a turmeric extract; and,
a hydroxy acid;
wherein the composition is applied onto skin comprising skin conditions selected from the group consisting of comprise psoriasis, severe dryness, itchiness, stretch marks, acne, and microbial infections.

28. The method of claim 27, wherein the turmeric extract comprises curcumin or turmerin.

29. The method of claim 27, wherein the hydroxy acid comprises glycolic acid in an amount ranging from about 0.1 weight percent to about 10 weight percent of the formulation.

30. The method of claim 27, wherein the formulation further comprises a vitamin B, aloe vera, or vitamin E in an amount ranging from about 0.1 weight percent to about 10 weight percent of the formulation.
